(19) United States
(12) Patent Application Publication
(10) Pub. No.: US 2025/0308670 A1
(43) Pub. Date: Oct. 2, 2025

(54) INTEGRATED THERAPY CHAIN

(71) Applicant: Aesculap AG, Tuttlingen (DE)

(72) Inventors: Michael Utz, Tuttlingen (DE); Ingrid Dupraz, Tuttlingen (DE); Thomas Grupp, Denkingen (DE); Allan Maas, Konstanz (DE)

(21) Appl. No.: 18/863,098

(22) PCT Filed: May 5, 2023
(86) PCT No.: PCT/EP2023/061981

§ 371 Date: Nov. 5, 2024

(30) Foreign Application Priority Data
May 6, 2022 (DE) 10 2022 111 285.3

(51) Int. Cl. G16H 20/40; A61F 2/46; G16H 10/60

(52) U.S. Cl. CPC G16H 20/40; A61F 2/461; G16H 10/60; A61F 2002/4633

(57) ABSTRACT

An integrated therapy chain is used for planning and carrying out insertion of an implant system. The therapy chain includes preoperative, intraoperative and postoperative therapy steps. The system includes a first data device for gathering, processing and providing patient-specific kinematic data, a second data device for gathering, processing and outputting patient data, a third data device for storing and outputting data of the predetermined implant system, and a control unit for controlling the data devices and for carrying out the therapy steps based on the aforementioned data. The first data device outputs a first data set including the currently gathered, patient-specific kinematic data, and a second data set including historical data based on older insertions of the implant system. The second data set supplements the first data set. A method can be performed for the preparation of an operation to insert an implant system.

### CROSS-REFERENCE TO RELATED APPLICATIONS

This application is the United States national stage entry of International Application No. PCT/EP2023/061981, filed on May 5, 2023, and claims priority to German Application No. 10 2022 111 285.3, filed on May 6, 2022. The contents of International Application No. PCT/EP2023/061981 and German Application No. 10 2022 111 285.3 are incorporated by reference herein in their entireties.

### FIELD

The present disclosure relates to a system of an integrated therapy chain for planning and carrying out insertion of a predetermined implant system, wherein the therapy chain comprises preoperative, intraoperative and postoperative therapy steps. Furthermore, the present disclosure relates to a method for preoperative preparation of surgery for insertion of an implant system.

### BACKGROUND

Today, only X-ray, CT or MRI images are used for surgical planning of a total endoprosthesis, in particular a total knee endoprosthesis. In most cases, a qualitative examination of the joint function is carried out by the attending physician beforehand.

Therefore, preoperative planning of this type of procedure is currently usually based solely on the patient's anatomical conditions or anatomy on the one hand and implant dimensions on the other hand. The weight restrictions of the patient defined by the manufacturer for special implant sizes have to be taken into account here. However, specifying a weight restriction as the sole parameter for selecting the implant is usually too one-sided and inaccurate. This often leads to complaints from the patient after the implant has been inserted, since the implant does not correctly and comprehensively meet the patient's requirements; in particular, the mechanical limits are often exceeded and can lead to serious complications later on. In other words, suboptimal alignment or a suboptimal implant type means that functionality is not/cannot be restored and can therefore lead to pain, in particular if there is no optimal adaptation to the patient's soft tissue situation.

It is therefore often a disadvantage that in most treatment cases the special features and limits of the implant system used cannot be taken into account. In particular, the interaction between the patient's circumstances, which cannot be influenced, and the implant system used is not considered in detail. This is due in particular to the fact that the doctor currently has no information available to assess whether his/her alignment technique, which appears to work well empirically, is mechanically safe and therefore the better or best solution in the long term. This is particularly relevant as not all patients need the same treatment, and some factors, in particular surgery and rehabilitation, can only be reacted to rather than acting in advance.

### SUMMARY

It is the objective technical object of the present disclosure to provide a system of an integrated therapy chain which essentially takes into account all, at least the calculable, contingencies in order to improve patient-specific care before, during and after surgery/intervention and to increase patient satisfaction. A further object is to eliminate or at least reduce the disadvantages of the prior art.

The preceding object is solved by an electronic system (device) with an integrated therapy chain for planning and carrying out insertion of a predetermined or selectable implant system, preferably for a knee endoprosthesis, shoulder endoprosthesis or hip endoprosthesis, wherein the therapy chain comprises preoperative, intraoperative and postoperative therapy steps. The selection of the implant system may be part of the therapy chain. Based on data, e.g. patient kinematics, implant limits, simulation and other boundary conditions, the best implant system for the patient and the best implant type for the patient can be selected. Alternatively, the term implant system may also refer to a cruciate ligament to be inserted, a tibial osteotomy or something similar.

The electronic system (device) according to the disclosure has a first data device for detecting, processing and providing patient-specific kinematic data, a second data device for detecting, processing and providing patient data, preferably a fourth data device for storing, processing and providing image material, a third data device for storing and providing data of the specific implant system/implant system selected from a number of implant systems, and a control unit for controlling the data devices and for performing the therapy steps based on the above data, wherein the first data device (2) is provided and configured to provide at least a first and a second data set, wherein the first data set comprises the currently detected patient-specific kinematic data, the second data set comprises historical data similar to the currently detected patient-specific kinematic data based on older insertions of the specific/selectable implant system, and the second data set is configured and provided to supplement the first data set with empirical values from the historical data.

In other words, the present disclosure relates to a technical/electronic solution for an integrated therapy chain for improving patient-specific care with a total endoprosthesis, preferably a knee endoprosthesis, as well as the subsequent rehabilitation measure. This solution acquires and takes into account preoperative as well as intraoperative and postoperative patient-specific data. Preoperatively, the choice of the optimal time for surgery may also be part of the therapy chain. The best time (not too early, but also not too late) may be determined based on the diagnosis, the degree of arthrosis, the patient's muscular and coordinative condition as well as other parameters. It is also possible to determine any beneficial pre-rehabilitation measures as preparation for the operation and to speed up postoperative rehabilitation.

This means that, in contrast to the state of the art and in the context of orthopaedic surgeries planning, patient-specific kinematics, demographic data (of the patient) and surgical results from previous, comparable cases are taken into account (computationally included) in order to also take into account the special features and limits of the implant system to be used. Against this background, an integrative approach is pursued in accordance with the present disclosure, in which the necessary data for or relating to the individual patient is detected, processed and provided step by step.

This means that the patient-specific information is supplemented/optimized/enriched with empirical values from previous, comparable cases. This involves examining which preoperative planning and intraoperatively implemented implant selection and implant position has achieved the best result in comparable patients to date. In addition, potential risks for the therapy (e.g. duration of anaesthesia) and the implant (e.g. body weight) resulting from the medical history are identified and options for minimizing risk are presented.

In addition to other data, this also includes the preoperative, intraoperative and postoperative detection of patient-specific kinematics using suitable methods known from the state of the art.

Further aspects of the present disclosure are described in more detail below.

It is advantageous if the preoperative, intraoperative and postoperative, patient-specific data can be combined, enriched/supplemented, and used for therapy optimization step by step across system limits. ‘Supplementable/enrichable’ means that the existing data is expanded or enriched with existing, in particular comparable, data in order to incorporate empirical values from previous cases into the planning and implementation of surgery.

It is preferred if the preoperative therapy steps comprise performing of an intelligent mobility analysis for detecting patient-specific kinematic data, planning as well as simulation of the insertion of the implant system, the intraoperative therapy step comprises performing of the insertion of the implant system, and the postoperative therapy step comprises rehabilitation.

It is advantageous if the preoperative therapy step of the intelligent mobility analysis, preferably gait analysis, is provided and configured to detect the patient-specific kinematic data as well as the patient data and process to it in the corresponding data devices.

It is advantageous if the first data device is provided and configured to determine kinematics and a phenotype from the patient-specific kinematic data. The patient-specific kinematic data may be used to derive the patient's phenotype, demographic data, and information about the patient's previous illnesses as well as their individual requirement profile, such as sport, everyday life and work activity.

It is preferred if the system has a fourth data device for storing, processing, and providing image material.

Here it is advantageous if the preoperative therapy step of the intelligent mobility analysis is provided and configured to also detect the image material and to process it in the corresponding data device.

It is preferred if the preoperative therapy step of the planning for insertion of the implant system is provided and configured to determine at least the implant type and the implant size based on the processed data of the first, second and fourth data devices and the data of the third data device.

In other words, the previous holistic view of the case is used for surgery planning with regard to implant selection and implant positioning.

It is advantageous if the fourth data device is provided and configured to determine a bone morphology based on the image material.

It is advantageous if the preoperative therapy step of the simulation for insertion of the implant system is provided and configured to test mechanical limits of the predetermined implant system based on the previous planning, the bone morphology, and an implant orientation.

In this subsequent simulation, the planning is checked, taking into account the available patient data and the mechanical limits of the planned implant system.

Here it is advantageous if the simulation is provided and configured to apply loads to the implant that correspond to the patient's kinematics, taking into account his/her body weight, anatomy, kinematics phenotype and the implant planning previously created by the physician, in order to approve the planning for intra-operative implementation if the mechanical limits of the implant system are not exceeded at any point in the loading cycle.

Otherwise, if the simulation shows that the mechanical limits have been exceeded at least once, it is preferable for the system to make suggestions for planning changes with the aim of complying with the mechanical limits of the implant system and, ideally, reconstructing the kinematics in such a way as to lead to the highest patient satisfaction for this phenotype.

It is preferred if the intraoperative therapy step of performing the insertion of the predetermined implant system is provided and configured to implement the planning based on the simulation using a navigation or robotic system.

In other words, the planning is then implemented intraoperatively using a navigation or robotic system. The planning may also be integrated as part of the navigation/robotic system. This has the effect that intraoperative conditions and, for example, the patient-specific soft tissue situation can be addressed in the simulation. An intraoperative change of the implant system may also be taken into account in this way.

It is advantageous if the postoperative therapy step for performing the rehabilitation can be adapted accordingly based on the patient-specific kinematic data of the first data security and a preoperative requirement profile. Based on the knowledge of the patient-specific kinematics and the preoperative requirement profile, the subsequent rehabilitation measures can be adapted accordingly. Using intelligent movement analysis, in particular gait analysis, rehabilitation may be tracked, supervised and individualized, even at a distance. The data from rehabilitation and the patient's postoperative kinematics may in turn be detected and used to optimize the further simulation model and preoperative planning. This allows the system to be continuously monitored and improved.

Furthermore, the present disclosure relates to a method for preoperative preparation of a surgery for insertion of an implant system, comprising the following steps:
detecting patient-specific data, in particular kinematic data, general patient data, and patient-specific image material (S1);
enriching the patient-specific data, in particular kinematic data, with comparative data;
determining a phenotype of the patient based on the patient-specific data, in particular the kinematic data;
performing planning regarding implant selection and positioning based on the enriched patient-specific data and the information on the implant system (S2); and
performing a simulation to test the planning with regard to mechanical limits of the implant system, taking into account the patient-specific data (S3);
approval of the preoperative planning if mechanical limits of the implant system have not been exceeded, or suggestions for plan modification in order to comply with the mechanical limits of the implant system.

The same procedure or the above system may also be applied to the treatment of other joints, such as the hip or shoulder. Furthermore, the described application may be extended by taking into account additional parameters, for example from other joints. For example, the musculoskeletal model of the knee may be extended to include bony structures of the ankle and hip or pelvis and the corresponding muscle forces.

Applications in the field of spinal surgery are also preferred. Here too, a musculoskeletal model of individual spinal segments or even the entire spine, including the pelvis if necessary, may be used for simulation.

Furthermore, in another embodiment, conservative treatments of joint problems without the use of implants may be considered. Examples include cruciate ligament reconstruction or tibial osteotomy. Particularly in the case of HTO (high tibial osteotomy), simulation of the correction angle may improve the long-term result.

In summary, one advantage of the system according to the disclosure is that preoperative/postoperative and intraoperative kinematics may be taken into account in order to find the best solution for each individual patient.

Finally, the present disclosure relates to a storage medium, on which the above method steps are stored.

### BRIEF DESCRIPTION OF THE DRAWING FIGURE

FIG. 1 is a representation illustrating a flowchart of the system according to an embodiment of the present disclosure.

### DETAILED DESCRIPTION

The following describes a preferred configuration example of the present disclosure based on the accompanying FIGURE.

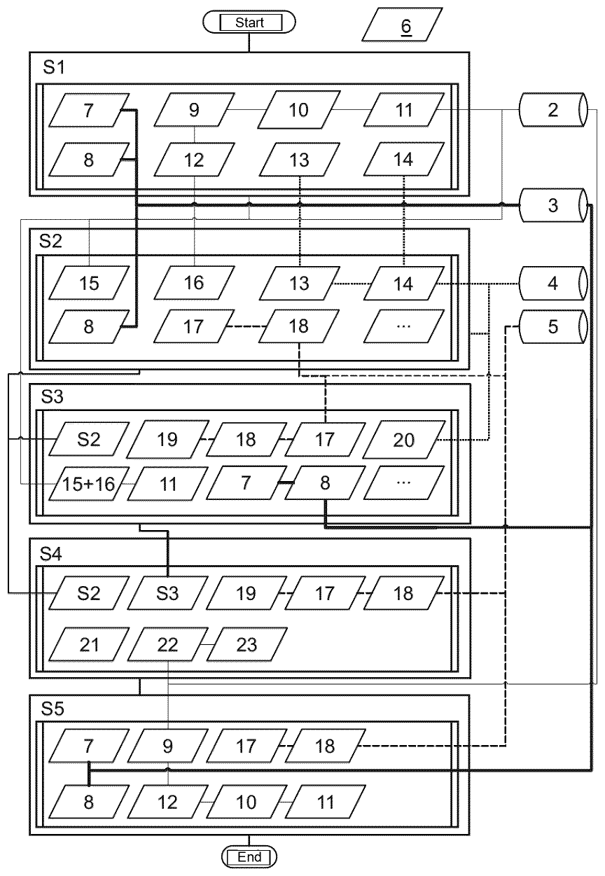

FIG. 1 is a representation illustrating a flowchart of the system according to a preferred configuration example of the present disclosure. In a first therapy step S1 of the preoperative preparation for performing insertion of a predetermined implant system, this step concerns detecting various data, in particular an intelligent mobility analysis, preferably an intelligent gait analysis.

In therapy step S1 as shown in FIG. 1, data about the patient 7 and demographics 8 are detected in a second data device 3. In a first data device 2, mobility analysis data 9, ground reaction forces 10, EMG data 11 and comparative data 12 are detected. In addition, X-ray images 13 and SSM (Statistic Shape Model) 14 are considered in therapy step S1. A “statistic shape model” is a static model of the bones that was calculated based on a plurality of CT data. The SSM can simulate any bone by varying the descriptive parameters or eigenvalues. This means that if the 3D SSM is adapted to the contour from the 2D X-ray image, a very good 3D reconstruction can be obtained from the 2D information.

In a second therapy step S2, which concerns the planning of the insertion of the implant system, data from the kinematics 15 and the phenotype 16 are used. The phenotype is derived from the data of the first therapy step S1 and are transferred directly to step S2 of the planning. The data relating to kinematics 15 corresponds to the data processed from the first data device 2 and which was detected in therapy step S1. Furthermore, therapy step S2 takes into account the data relating to demographics 8, which can be obtained both from the second data device 3 and directly from the first therapy step S1. The image material 13 and 14 from therapy step S1 is transferred to therapy step S2 and is stored and processed in a fourth data device 4. In step S2 of the planning, data about the implant system is also relevant. Thus, corresponding data of an implant type 17 and an implant size 18 are taken into account and are stored in a third data device 5.

In a third preoperative therapy step S3, which corresponds to the simulation of the insertion of the implant system, data relating to the kinematics 15, the phenotype 16, and the EMG data 11 are used from the first data device 2. Data relating to the patient 7 and demographics 8 are taken into account from the second data device 3. In the fourth data device 4, the image materials 13 and 14 are processed to provide the third therapy step S3 with a bone morphology 20. The simulation S3 takes into account the aspects of the planning S2 as well as an implant orientation 19, the implant type 17 and the implant size 18.

The S3 simulation provides information on the interaction of patient conditions based on the patient-specific kinematic data and the mechanical limits of the planned implant system to be inserted.

The therapy step S4 is an intraoperative step. In S4, the implant system with the aspects of the implant type 17, the implant size 18, and the implant orientation 19 is inserted or the surgery is performed using the planning S2 and the simulation S3 via a navigation or robotic system. The first data device 2 provides data of postoperative kinematics 22 and intraoperative kinematics 23 to step S4. Furthermore, data relating to the soft tissue 21 of the patient is taken into account in step S4.

A postoperative therapy step S5 takes into account the data on the patient 7 and demographics 8 from the second data device 3 as well as data on the patient-specific kinematics 9, 10, 11 and 12 and the implant type 17 and implant size 18 for rehabilitation measures. The patient-specific kinematic data for therapy step S5 is derived from the kinematic data detected in therapy step S1, which is provided by the first data device 2, and the post- and intraoperative kinematics 22 and 23 used in therapy step S4.

### LIST OF REFERENCE SIGNS

1 integrated therapy chain
2 first data device
3 second data device
4 fourth data device
5 third data device
6 control unit
7 patient
8 demography
9 mobility analysis data
10 ground reaction forces
11 EMG data
12 historical data
13 X-ray image
14 SSM
15 kinematics
16 phenotype
17 implant type
18 implant size
19 implant orientation
20 bone morphology
21 soft tissue
22 postoperative kinematics
23 intraoperative kinematics

1.-12. (canceled)

13. A system of an integrated therapy chain for planning and carrying out insertion of an implant system, which is a specific or selectable implant system, the integrated therapy chain having a preoperative therapy step, an intraoperative therapy step and a postoperative therapy step, the system comprising:
a first data device for detecting, processing and providing patient-specific kinematic data;
a second data device for detecting, processing and providing patient data;
a third data device for storing and providing data of the implant system; and
a control unit for controlling the first data device, the second data device and the third data device, and for performing the preoperative therapy step, the intraoperative therapy step and the postoperative therapy step based on the patient-specific kinematic data, the patient data, and the data of the implant system,
the first data device being configured to provide at least a first data set and a second data set,
the first data set comprising the patient-specific kinematic data,
the second data set comprising historical data similar to the patient-specific kinematic data based on older or earlier insertions of the implant system,
the second data set configured to enrich the first data set with empirical values from the historical data,
the preoperative therapy step comprising a detection of the patient-specific kinematic data, a planning of an insertion of the implant system, and a simulation of the insertion of the implant system,
the intraoperative therapy step comprising inserting the implant system, and
the postoperative therapy step comprising a rehabilitation,
the planning of the insertion of the implant system configured to determine implant selection and positioning based on an enriched patient-specific kinematic data and information on the implant system, and
the simulation of the insertion of the implant system configured to test mechanical limits of the implant system, taking into account the patient-specific kinematic data and the patient data.

14. The system according to claim 13, wherein the preoperative therapy step further comprises choosing an optimal time for surgery.

15. The system according to claim 13, wherein the preoperative therapy step is configured to detect and process the patient-specific kinematic data in the first data device, and detect and process the patient data in the second data device.

16. The system according to claim 15, wherein the first data device is configured to determine kinematics and a phenotype from the patient-specific kinematic data.

17. The system according to claim 13, wherein the system comprises a fourth data device for storing, processing, and providing image material.

18. The system according to claim 17, wherein the preoperative therapy step is further configured to detect the image material and process the image material in the fourth data device.

19. The system according to claim 18, wherein the preoperative therapy step is configured to determine at least an implant type and an implant size based on processed data of the data devices and the data of the third data device.

20. The system according to claim 19, wherein the fourth data device is configured to determine a bone morphology based on the image material.

21. The system according to claim 20, wherein the preoperative therapy step is configured to test mechanical limits of the implant system based on the planning of the insertion of the implant system, the bone morphology, and an implant orientation.

22. The system according to claim 21, wherein the intraoperative therapy step is configured to implement the planning of the insertion of the implant system, based on the simulation of the insertion of the implant system, using a navigation or robotic system.

23. The system according to claim 22, wherein the rehabilitation is adaptable based on the patient-specific kinematic data and a preoperative requirement profile.

24. A method for preoperative preparation of a surgery for insertion of an implant system into a patient, the method comprising the following steps:
detecting patient-specific kinematic data and patient data;
enriching the patient-specific kinematic data with comparative data to generate enriched patient-specific kinematic data, the comparative data comprising historical data similar to the patient-specific kinematic data based on older or earlier insertions of the implant system;
determining a phenotype of the patient based on the patient-specific kinematic data;
performing planning regarding implant selection and positioning based on the enriched patient-specific kinematic data and information on the implant system;
performing a simulation to test the planning with regard to mechanical limits of the implant system, taking into account the patient-specific kinematic data and the patient data; and
one of:
approving the planning when the mechanical limits of the implant system have not been exceeded, or
suggesting a plan modification to comply with the mechanical limits of the implant system.
